(19) United States
(12) Patent Application Publication
(10) Pub. No.: US 2004/0192660 A1
(43) Pub. Date: Sep. 30, 2004

(54) Protocol for improving vision

(76) Inventor: John P. Mullally, Gratiot, MI (US)

Correspondence Address: Arnold S. Weintraub, The Weintraub Group, P.L.C., Suite 240, 32000 Northwestern Highway, Farmington Hills, MI, 48334, US

(21) Appl. No.: 10/820,264

(22) Filed: Apr. 8, 2004

Related U.S. Application Data
(63) Continuation-in-part of application No. 10/798,017, filed on Mar. 11, 2004.
(60) Provisional application No. 60/461,534, filed on Apr. 9, 2003.
(60) Provisional application No. 60/453,917, filed on Mar. 12, 2003.
(60) Provisional application No. 60/482,574, filed on Jun. 24, 2003.

(51) Int. Cl. A61K 31/573; A61K 31/415; A61K 31/445

(52) U.S. Cl. 514/171; 514/406

(57) ABSTRACT

A method and composition for reducing highly sensitive C-reactive protein to improve the vision of a user is achieved through the daily administration of a leukotriene inhibitor, and antihistamine and a corticosteroid. The composition may be administered singly or as a single medicament. Typically, the leukotriene inhibitor and antihistamine are administered orally and the steroid nasally infused.

### CROSS-REFERENCE TO RELATED APPLICATIONS

This application is a continuation-in-part application of co-pending U.S. patent application Ser. No. 10/798,017 filed on Mar. 11, 2004 entitled “Composition and Method for Treating Inflammations” which, in turn, is a completion application of Provisional Patent Application Serial No. 60/461,534 filed on Apr. 9, 2003, the disclosures of which are hereby incorporated by reference.

### FIELD OF THE INVENTION

The present invention pertains to medical protocols. More particularly, the present invention pertains to medical protocols for treating vision. Even more particularly, the present invention concerns medical protocols for improving vision in persons having diabetes.

### PRIOR ART

In the above-referred to co-pending application, the disclosure of which is hereby incorporated by reference, there is disclosed therein a medical protocol for lowering C-reactive protein in the body of user. According to the protocol defined therewithin, it is theorized that by eliminating sinus drainage the bacteria and viruses which are normally captured therewithin and which ultimately enter into bloodstream are eliminated through the administration of a leukotriene inhibitor, an antihistamine and a steroid. Levels of highly sensitive C-reactive protein are reduced in the body of the user, thus, relieving systemic inflammation. The principal component of the protocol is the leukotriene inhibitor. However, by the administration of all three of the components of the protocol, dramatic decreases in the C-reactive protein in the system of a user has been effected thereby providing relief from a variety of systemic inflammations.

It has now been observed that by lowering C-reactive protein levels in the body of a user, there is a concomitant improvement in the vision of persons, and in particular, and those suffering from diabetes. Thus, the present invention adopts and adapts the protocol of the above-referred to co-pending application in improving the vision of a user.

### SUMMARY OF THE INVENTION

In accordance with the present invention, there is provided a protocol for improving the vision of a user by the lowering of C-reactive protein within the body of such of a user. The protocol hereof, generally, comprises the administering to a user a daily dosage of: (a) a leukotriene inhibitor, (b) an antihistamine, (c) a corticosteroid, and (d) mixtures thereof.

In a second aspect hereof there is provided a composition for reducing C-reactive protein for improving the vision of a user which comprises an admixture of a leukotriene inhibitor, an antihistamine and a corticosteroid.

The composition is administered on a daily basis as a preventative treatment.

For a more complete understanding of the present invention reference is made to the following detailed description and accompanying non-limitative examples.

### DESCRIPTION OF THE PREFERRED EMBODIMENTS

As hereinabove noted, the present invention comprises, in a first aspect, a method for reducing and/or eliminating C-reactive protein in the body to improve the vision of a user by administering a composition selected from the group consisting of: (a) a leukotriene inhibitor, (b) an antihistamine, (c) a corticosteroid, and (d) mixtures thereof.

In a second aspect hereof there is provided a composition for improving the vision of a user by reducing C-reactive protein which comprises an admixture of at least two of and, preferably, each of: (a) a leukotriene inhibitor, (b) an antihistamine, and (c) a corticosteroid.

In accordance with the present invention it has been found that a daily treatment of the above-noted composition is effective in reducing highly sensitive C-reactive protein and the concomitant vision improvement.

Generally, the leukotriene inhibitor is administered in a dosage from about 1 to 20 milligrams on a daily basis and, preferably, from about 5 to about 15 milligrams. The leukotriene inhibitor may be ingested as a pill, capsule, as a liquid, etc.

Similarly, the antihistamine is ingested, orally or nasally, on a daily basis and in an amount ranging from about 50 to about 250 milligrams and, preferably, from about 175 to about 200 milligrams daily.

The corticosteroid is usually found in a liquid transport or delivery medium, such as a nasal spray or the like and ordinarily, a minimal amount ranging from about 110 μcg to about 220 μcg as obtained from about 1 to about 4 nasal sprays is effective once or more daily.

In using this treatment, it is preferred where all three medicaments are used and that the leukotriene inhibitor and the antihistamine be administered each as a pill or gelcap while the steroid, as noted, is infused as a nasal spray.

Typical of the leukotriene inhibitors are those which are selected from the group consisting of albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and the like, as well as mixtures thereof.

The inflammation reducing leukotriene inhibitor may be any of those commercially available leukotriene inhibitors such as “Zyflo®” (zileuton), “Accolate®” (zafirlukast), and “Singulair®” (a montelukast sodium) each sold commercially and available in pill form. Preferably, the leukotriene inhibitor is the montelukast sodium, which is sold commercially in pill form under the trademark “SINGULAIR®.”

The antihistamine can be any of those which are commercially available such as those sold under the name “Zyrtec®” (cetirizine), “Allegra®” (fexofenadine), “Claritin®” (loratadine), and Clarinex®.

The nasal steroid is, preferably, either azelastine or fluticasone propionate. This propionate is sold commercially under the name “Flonase®.” The aselastine is sold commercially under the name “Astelin®.” Other useful nasal steroids are those sold commercially under various trademarks such as, for example, “Nasonex®” (mometasone furoate monohydrate), “Nasacort AQ®” (triamcinolone acetoniode), and “Rhinocort Aqua®” (budesonide), to name a few.

According to the invention, a preferred dosage or “composition” to improve vision includes at least two of and, preferably, all three of: (a) a capsule of Singulair®, (b) one capsule of Allegra®, and (c) a prescribed infusion the Astelin® or Flonase® into each nasal passage. By taking the daily dosages, it is possible to produce highly sensitive C-reactive protein and, thus, improve vision.

Although not wishing to be bound any theory, it is believed that the inflammation of the sinuses and nasal pathways is transmitted systemically through a bacteria, virus, fungus or an immune response associated with sinus drainage that is, then, transmitted through the bloodstream. It is believed that this theory also applies to other pathways including arteries, veins, and to some degree, body organs. What the present invention contemplates is the blocking of the inflamed pathways and, thus, treating the condition.

It is contemplated that the medicaments defined herein be administered at one time. It is also contemplated and within the scope hereof, that a single compound, as a liquid vehicle, be administered with the requisite amounts. In other words, there would be provided as a composition a sprayable compound having the leukotriene inhibitor, the antihistamine, and the corticosteroid all suspended in a liquid vehicle or non-toxic delivery system which can then be administered either orally or nasally through a spray and transmitted either through the mouth or the nasal passages.

Alternatively, it is possible to provide a capsule, pill or gelcap at least two or, preferably, containing all of the three components hereof.

In attempting to appreciate the benefits of the present invention, it is worthy to note that bacteria, fungi, and viruses all colonize in the sinus cavity. By attacking the source those diseases which are attributable to elevated high sensitive C-reactive protein levels in the blood are, necessarily, dramatically reduced and/or eliminated.

The following is an illustrative example showing the benefits of the present invention:

### EXAMPLE

A 47-year-old, female, diabetic patient was examined and initially determined to have the following vision using a standard eye chart:

| | |
|---|---|
| Right eye | 20/150 |
| Left eye | 20/100 |

The patient wore corrective lenses, but still experienced difficulty in reading. The patient was placed on the protocol of one daily capsule dosage of the Singulair® leukotriene inhibitor; one daily capsule dosage of the Allegra® antihistamine; and two daily infusions of from 1 to 4 squeezes of the Astelin® azelastine steroid from a squeeze bottle.

After five weeks of administering the protocol on a daily basis, the measured vision on a standard eye chart was:

| | |
|---|---|
| Right eye | 20/30 |
| Left eye | 20/40 |

with an ability to read and recognize words without prescription lenses.

Because of the nature of the medicaments, i.e. all over-the-counter compositions, they can be administered to most persons safely and, typically, without interference with other medicines which may be taken until the full effects of the present invention are realized.

From the above it is to be readily appreciated that there has been described herein a medical composition and method of use therefor which improves the vision of a user.

1. A method for improving the vision of a user which comprises:
administering on a daily basis for a period of at least about 2 days, a composition selected from the group consisting of:
(a) a leukotriene inhibitor,
(b) an antihistamine,
(c) a corticosteroid, and
(d) mixtures thereof.
wherein highly sensitive C-reactive protein levels in the body of a user are reduced to improve the vision thereby.

2. The method of claim 1 wherein the selected composition is used in an amount of:
(a) from about 1 to about 20 milligrams of leukotriene inhibitor,
(b) from about 50 to about 250 milligrams of antihistamine, and
(c) from about 110 μcg to about 220 μcg of corticosteroid.

3. The method of claim 2 wherein the selected composition is used in an amount of:
(a) from about 5 to about 15 milligrams of the leukotriene inhibitor,
(b) from about 175 to about 200 milligrams of the antihistamine, and
(c) from about 110 μcg to about 220μcg of the corticosteroid.

4. The method of claim 2 wherein the leukotriene inhibitor is selected from the group consisting of:
albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and mixtures thereof.

5. The method of claim 2 wherein the antihistamine is selected from the group consisting of:
cetirizine, fexofenadine and lortadine.

6. The method of claim 2 wherein the steroid is selected from the group consisting of:
mometasone furoate mononhydrate, triamcinalone, acetoniode, budesonide and azelastine.

7. The method of claim 2 wherein:
(a) the leukotriene inhibitor is montelukast sodium,
(b) the antihistamine is selected from the group consisting of cetirizine, fexofenadine and loratadine, and
(c) the steroid is selected from the group consisting of azelastine and fluticosone propionate.

8. The method of claim 2 wherein the composition comprises:
(a) the leukotriene inhibitor,
(b) the antihistamine, and
(c) the corticosteroid.

9. The method of claim 2 wherein:
the leukotriene and the antihistamine are administered orally and the steroid is nasally infused.

10. A composition for reducing C-reactive protein to improve the vision of a user, consisting essentially of:
(a) a leukotriene inhibitor,
(b) an antihistamine, and
(c) a corticosteroid.

11. The composition of claim 10 wherein the composition comprises:
(d) from about 1 to about 20 milligrams of the leukotriene inhibitor,
(e) from about 150 to about 250 milligrams of antihistamine, and
(f) from about 110 μcg to about 220 μcg of corticosteroid.

12. The composition of claim 11 wherein the composition comprises:
(d) from about 5 to about 15 milligrams of leukotriene inhibitor,
(e) from about 175 to about 200 milligrams of antihistamine, and
(f) from about 110 μcg to about 220 μcg of corticosteroid.

13. The composition of claim 11 wherein the leukotriene inhibitor is selected from the group consisting of:
albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and mixtures thereof.

14. The method of claim 2 wherein the antihistamine is selected from the group consisting of:
cetirizine, fexofenadine and lortadine.

15. The composition of claim 11 wherein the steroid is selected from the group consisting of:
(a) mometasone furoate mononhydrate,
(b) triamcinalone,
(c) acetoniode,
(d) budesonide, and
(e) azelastine.

16. The composition of claim 11 wherein:
(a) the leukotriene is montelukast sodium,
(b) the antihistamine is cetirizine, fexofenadine and loratadine, and
(c) the steroid is azelastine.

17. The composition of claim 16 wherein:
the leukotriene and the antihistamine are administered orally and the steroid is nasally infused.

18. A method for improving the vision of a user by lowering C-reactive protein levels in the body thereof which comprises:
administering on a daily basis a composition selected from the group consisting of:
(a) a leukotriene inhibitor,
(b) an antihistamine,
(c) a corticosteroid, and
(d) mixtures thereof.

19. The method of claim 18 wherein:
the composition is a mixture of the inhibitor, the antihistamine, and the corticosteroid.

20. The method of claim 19 wherein:
(a) the leukotriene inhibitor is a montelukast sodium, the inhibitor being used in an amount ranging from about 5 to about 15 milligrams,
(b) the antihistamine is selected from the group consisting of cetirizene, fexofenadine and lortadine, the antihistamine being used in an amount ranging from about 175 to about 200 milligrams, and
(c) the steroid is selected from the group consisting of:
momeasone furoate monohydrate, triamcinalone, acetoniode, budesonide, and azelastine, the steroid being used in an amount ranging from about 110 μcg to about 220 μcg.
